SEQUENCE LISTING (10 sequences)

SEQ ID NO: 1 - DNA, 624 bp, Artificial Sequence
  Nucleotide sequence encoding for heavy chain of the variable region of 2E7
atgctggtggagtctgggggaggcttggtgcaacctggaggatccatgaaactctcctgtatagtctcgggatttactttcagttattattggatgtcttgggtccgccagtctccagagaaggggtttgagtgggttgctgaaatcagattgaaatctgagaattatgcaacaaattatacggagtctgtgaaagggaagttcaccatctcaagagatgattccaaaagtcgtctctacctgcaaatgaacagcttaggagctgaggacactggaatttattactgtctaactggttatgcctggtttgcttattggggccaagggactctagtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaag

SEQ ID NO: 2 - DNA, 531 bp, Artificial Sequence
  Nucleotide sequence encoding for light chain of the variable region of 2E7
gacgtccagatgatccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaacatacacattttcccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 3 - protein, 208 aa, Artificial Sequence
  Heavy chain of the variable region of 2E7
MLVESGGGLVQPGGSMKLSCIVSGFTFSYYWMSWVRQSPEKGFEWVAEIRLKSENYATNYTESVKGKFTISRDDSKSRLYLQMNSLGAEDTGIYYCLTGYAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTK

SEQ ID NO: 4 - protein, 177 aa, Artificial Sequence
  Light chain of the variable region of 2E7
DVQMIQSPKRLIYLVSKLDSGVPDRFTGSGSGTDFTLKISRVEAEDLGVYYCVQHTHFPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 21 bp, Artificial Sequence
  Forward primer for nucleotide sequence encoding for heavy chain of the variable region of 2E7
atgctggtggagtctggggga

SEQ ID NO: 6 - DNA, 21 bp, Artificial Sequence
  Forward primer for nucleotide sequence encoding for heavy chain of the variable region of 2E7
aagctggtggaatctggagga

SEQ ID NO: 7 - DNA, 21 bp, Artificial Sequence
  Reverse primer for nucleotide sequence encoding for heavy chain of the variable region of 2E7
cttggtgctgctggccgggtg

SEQ ID NO: 8 - DNA, 24 bp, Artificial Sequence
  Forward primer for nucleotide sequence encoding for light chain of the variable region  of 2E7
ccgtttgatttccagcttggtgcc

SEQ ID NO: 9 - DNA, 24 bp, Artificial Sequence
  Forward primer for nucleotide sequence encoding for light chain of the variable region of 2E7
ccgtttcagctccagcttggtccc

SEQ ID NO: 10 - DNA, 24 bp, Artificial Sequence
  Reverse primer for nucleotide sequence encoding for a light chain of the variable region of 2E7
gacattgagctcacccagtctcca

(19) United States
(12) Patent Application Publication
(10) Pub. No.: US 2017/0342136 A1
(43) Pub. Date: Nov. 30, 2017

(54) MONOCLONAL ANTIBODY AGAINST MURAMYL PEPTIDES IN PREVENTION AND TREATMENT OF IMMUNE-MEDIATED DISEASES

(71) Applicant: Agency for Science, Technology and Research, Singapore (SG)

(72) Inventors: Yue Wang, Singapore (SG); Zhenxing Huang, Singapore (SG)

(21) Appl. No.: 15/538,077

(22) PCT Filed: Jan. 12, 2016
(86) PCT No.: PCT/SG2016/050013

§ 371 Date: Jun. 20, 2017

(30) Foreign Application Priority Data
Jan. 12, 2015 (SG) 10201500223V

(51) Int. Cl. C07K 16/12; A61K 39/085; A61K 39/40; A61K 39/00

(52) U.S. Cl. CPC C07K 16/12; A61K 39/40; A61K 39/085; C07K 2317/33; A61K 2039/505; C07K 2317/76; C07K 2317/92; A61K 2039/545

(57) ABSTRACT

Disclosed are methods of treating an autoimmune or inflammatory disease using a composition comprising an isolated antibody or an antigen-binding fragment or variant thereof that is capable of binding to muramyl peptide, derivative, analog or salt thereof, wherein said muramyl peptide comprises muramic acid and an amino acid selected from the group consisting of alanine, isoglutamine, glutamic acid and a salt thereof. In one preferred embodiment, the composition can comprise of the isolated antibody or an antigen-binding fragment and one or more therapeutic agents such as Tumor Necrosis Factor (TNF) inhibitor. The autoimmune or inflammatory disease is selected from a group consisting of sepsis, septic shock, Crohn's disease, rheumatoid arthritis, asthma, allergy, atopic disorders, multiple sclerosis, pertussis, gonorrhea, inflammatory bowel disease, and antibiotic associated disorder.

### CROSS-REFERENCE TO RELATED APPLICATIONS

This application claims the benefit of priority of Singapore Patent Application No. 10201500223V, filed on 12 Jan. 2015, the contents of it being hereby incorporated by reference in its entirety for all purposes.

### FIELD OF THE INVENTION

The present invention generally relates to the field of immunology and immune-mediated disease. In particular, the present invention relates to antibodies, compositions containing the antibodies, and the use of the antibodies and compositions in the prevention and treatment of diseases.

### BACKGROUND OF THE INVENTION

All bacteria contain peptidoglycans as the main cell wall constituent. Peptidoglycans, which are formed from subunits of muramyl peptides, undergo cycles of assembly and disassembly required for cell wall remodeling during cell growth and division. Muramyl peptides are generated during the disassembly and recycled to construct new peptidoglycans during cell growth and septation. Accordingly, muramyl peptides are constantly released from many bacterial species during proliferation. They are also generated and released when bacterial cells are lysed by phages, antibiotics and host phagocytes.

The human microbiota, which contains 10 to 100 trillion non-pathogenic bacterial cells, constantly produces and secretes large quantities of muramyl peptides. A fraction of these molecules can enter the host circulation system via various mechanisms during the homeostatic state of the host as well as under numerous pathophysiological conditions. Many muramyl peptides are potent signaling molecules and have been shown to strongly influence multiple physiological processes in the human host. Biological activities that have been linked to muramyl peptides include adjuvanticity, somnogenicity, pyrogenicity, and toxicity to ciliated epithelial cells.

Muramyl peptides impact the human physiology by binding to specific peptidoglycan recognition proteins (PGRPs). Humans have four PGRPs, which are able to directly bind to both Gram-positive and Gram-negative peptidoglycan, and two intracellular peptidoglycan sensors, Nod1 and Nod2 belonging to a large family of pattern recognition receptors that recognize conserved microbe- or pathogen-associated molecular patterns. Nod2 recognizes the muramyl dipeptide N-acetylmuramyl-L-alanyl-D-glutamine, while Nod1 recognizes the D-γ-glutamyl-mDAP motif in the peptide. Both Nod1 and Nod2 trigger and regulate the host immune response by activating the transcription factor NF-κB, which in turn switches on the production of proinflammatory cytokines and chemokines and expression of other defense genes. Nod1, Nod2 and NF-κB have been implicated in numerous human diseases, in particular immune-mediated diseases such as sepsis, septic shock, Crohn's disease, rheumatoid arthritis, asthma, allergy, atopic disorders, multiple sclerosis, pertussis, gonorrhea, inflammatory bowel disease, and antibiotic-associated disorder. These immune-mediated diseases may afflict any organ system and result in significant morbidity, reduced quality of life and even premature death.

Treatment of immune-mediated diseases generally involves attempts to control the progress of the diseases and also to reduce the severity of the symptoms. For example, treatment of rheumatoid arthritis usually involves the use of non-steroidal anti-inflammatory agents (NSAIDs), corticosteroids, and disease modifying anti-rheumatic drugs (DMARDs). NSAIDs are mainly used to reduce acute inflammation thereby decreasing pain and improving physical function of the affected joints. Corticosteroids have both anti-inflammatory and immunoregulatory activities. However, prolonged use of corticosteroids has numerous side effects, some of which may be severe. DMARDs have been shown to alter the disease course and improve radiographic outcome, however, it generally takes much longer for DMARDs to take effect. Therefore, NSAIDs and corticosteroids are often used as temporary adjunctive therapy while waiting for DMARDs to exert their anti-inflammatory effects.

Currently there are a number of DMARDs available on the market. Therapeutic biologics, a subset of DMARDs, are known to have a more rapid onset of effect compared to traditional (non-biologic) DMARDs. Since therapeutic biologics are “targeted therapies” that target specific proteins known to be involved in the pathogenesis of a disease, they are known to be associated with less side effects as compared to traditional DMARDs that affect the entire immune system. However, many patients with rheumatoid arthritis do not respond well to the currently available therapeutic biologics. Since immune-mediated diseases such as rheumatoid arthritis affect a large percentage of the population (for example, rheumatoid arthritis affects approximately 1% of the adult population worldwide), there is a need to provide alternative biologics for the treatment of immune-mediated diseases which overcome, or at least ameliorate, one or more of the disadvantages described above.

### SUMMARY OF THE INVENTION

In a first aspect, there is provided a method of prophylactically or therapeutically treating an autoimmune or inflammatory disease comprising administering an isolated antibody or an antigen-binding fragment thereof, wherein the isolated antibody or antigen-binding fragment thereof is capable of binding to a muramyl peptide, or a derivative or an analog or a salt thereof, wherein the muramyl peptide comprises muramic acid and an amino acid selected from the group consisting of alanine, isoglutamine, glutamic acid, and a salt thereof.

In a second aspect, there is provided an isolated antibody or an antigen-binding fragment thereof as defined in the first aspect, for use in the prophylactic or therapeutic treatment of an autoimmune or inflammatory disease.

In a third aspect, there is provided use of an isolated antibody or an antigen-binding fragment thereof as defined in the first aspect in the manufacture of a medicament for prophylactically or therapeutically treating an autoimmune or inflammatory disease.

In a fourth aspect, there is provided a composition comprising an isolated antibody or an antigen-binding fragment thereof as defined in the first aspect, one or more therapeutic agents, and optionally a pharmaceutically acceptable carrier.

In a fifth aspect, there is provided a method of prophylactically or therapeutically treating an autoimmune or inflammatory disease comprising administering a composition of the fourth aspect.

In a sixth aspect, there is provided a composition of the fourth aspect for use in the prophylactic or therapeutic treatment of an autoimmune or inflammatory disease.

In a seventh aspect, there is provided use of the composition of the fourth aspect in the manufacture of a medicament for prophylactically or therapeutically treating an autoimmune or inflammatory disease.

### BRIEF DESCRIPTION OF THE DRAWINGS

The invention will be better understood with reference to the detailed description when considered in conjunction with the non-limiting examples and the accompanying drawings, in which:

FIG. 1 shows the determination of the Kd value of one example of an antibody as described herein, which is the monoclonal antibody 2E7. To determine the Kd of 2E7 to N-acetylmuramyl-L-alanyl-D-glutamine, the bottom of wells of a 96-well plate was coated with MDP-OVA. 2-fold serial dilutions of 2E7 were added to the wells for incubation. After washes to remove unbound Abs, an anti-mouse-IgG Ab coupled with horseradish peroxidase (HSP) was added. After incubation, unbound secondary Ab was removed by washes and a chromogenic substrate of HSP was added. After reaction, the color intensity in each well was measured using a microtiter plate reader at 492 nm. A binding curve was generated using CurveExpert Basic program (http://www.curveexpert.net) by fitting 2E7 concentrations and OD492 values to the Weibull Model equation y=a−b*exp(−C*x̂d) using CurveExpert Basic. r: correlation coefficient; s: standard error. Coefficient data: a=2.8768241, b=2.8055033, c=212.44541, and d=0.8659833. Y₅₀=1.45, X=0.00131 μg/ml. Kd=0.0131/[150×10⁹]=8.7 pM (IgG MW=150).

FIG. 2 (A) shows a standard curve that was generated by fitting N-acetylmuramyl-L-alanyl-D-isoglutamine concentrations and OD₄₉₂ values to the Shifted Power Fit equation y=a*(x−b)̂c using CurveExpert Basic. The standard curve was used to determine the peptidoglycan concentration in a sample. (B) shows a graph showing Staphylococcus aureus and Escherichia coli growth in liquid LB medium in the presence or absence of amoxicillin. Both bacteria were grown to OD₆₀₀=1.5. Each culture was divided into two, and one treated with 40 μg/ml amoxicillin (Amx) and the other untreated. Aliquots were collected at the indicated times and the amount of MPs in the supernatant was determined by competitive ELISA. (C) shows fluorescence microscope images of Staphylococcus aureus and Escherichia coli after incubation with 2E7. Both Staphylococcus aureus and Escherichia coli live cells were first washed with phosphate buffered saline (PBS) 3 times and then incubated with 2E7. After removing unbound antibodies by PBS washes, the cells were incubated with a FITC-labeled anti-mouse IgG Ab before fluorescence microscopic examination. FIG. 2 demonstrates that 2E7, which is an example of the antibody of the present disclosure, recognizes natural peptidoglycan.

FIG. 3 (A) shows a bar graph of the results of ELISA on the study of amoxicillin administration into mice. Amoxicillin was administered orally to 50 mice at 12-h intervals and three mice were sacrificed every 4 h to collect blood for peptidoglycan quantification by ELISA. (B) shows a bar graph of the results of ELISA on blood samples from a human patient who received multiple IV injections of Augmentin. Serial blood samples were taken. Time for blood draw and IV injection are indicated. FIG. 3 demonstrates that amoxicillin causes a significant increase in blood level of MPs in mice and humans.

FIG. 4 shows the detection and quantification of PG subunits in healthy donors. Blood was taken from seven generally healthy donors and the level of peptidoglycan in the serum was determined by competitive ELISA using E2F. The sex and age of the donors are shown.

FIG. 5 shows the use of an implantable osmotic pump to achieve continuously elevated serum muramyl dipeptides (MDPs) level in mouse. (A) illustrates the subcutaneous implantation of an osmotic pump loaded with 200μ1 of 5 mg/ml MDP and released at a rate of 0.25μ1/hr onto the back of a mouse. (B) shows the MDP levels in the serum collected from the mice at 0 day, 3 days, 10 days, 17 days and 24 days after the implantation, measured by competitive ELISA.

FIG. 6 demonstrates that increased levels of MDP promoted the development of rheumatoid arthritis in mouse. (A) shows the Collagen Induced Arthritis (CIA) mouse model. DBA/1J mice were induced to develop arthritis by immunization with an emulsion of complete Freund's adjuvant and type II collagen, and each mouse also carried an implanted pump as described in FIG. 5 that releases either PBS or MDP at 0.25 μl/hr. Upper panel shows pictures of the hind paws of the mice. Lower panel shows sections of the hind paws stained with haematoxylin and eosin (H&E) to visualize the joint tissues (b: bone; c: cartilage). (B) is a graph showing that increase in MDP concentration resulted in increased clinical paw scores of rheumatoid arthritis progression of the CIA mice with an implanted pump releasing the indicated solutions in a time dependent manner, (n=4).

FIG. 7 demonstrates the effect of 2E7 in the prevention of the onset of rheumatoid arthritis. (A) shows the Collagen Antibody Induced Arthritis (CAIA) mouse model. Balb/c mice were stimulated to develop arthritis by injection with a cocktail of anti-collagen monoclonal antibodies. Upper panel shows representative pictures of paws of the CAIA mice at the end of the experiment. Each of the CAIA mice was given a single dose of intraperitoneal (IP) injection with 2E7 or an isotype control antibody at the onset of rheumatoid arthritis. Lower panel shows sections of paws of the CAIA mice stained with H&E to show the joint tissues. (B) is a graph showing that treatment with 2E7 significantly prevented the onset of rheumatoid arthritis as compared to the isotope control.

FIG. 8 demonstrates the dose-dependent therapeutic effects of 2E7 on rheumatoid arthritis, by showing the effects of 2E7 on clinical paw score at doses of 160 mg/kg, 40 mg/kg and 10 mg/kg in the mouse CAIA model. CAIA mice were treated by a single IP injection with 2E7 or the control antibody (Ctrl) at indicated doses (n=5).

FIG. 9 demonstrates that 2E7 treats rheumatoid arthritis by neutralizing circulating PGNs. (A) shows a reduced clinical paw score in CAIA mice by the administration of 2E7 at the onset of rheumatoid arthritis, compared to a control antibody. (B) shows an increased clinical paw score upon administration of 2E7 with MDP compared to administration of 2E7 alone. n=8, p<0.01. The results show that introducing more MDP into the circulation will reduce or even block the effect of 2E7, thereby providing evidence that 2E7 prevents the progression of rheumatoid arthritis by neutralizing MDP in the circulation.

FIG. 10 shows the effect of 2E7 in the prevention of the relapse of rheumatoid arthritis. Mice were assigned to 2E7 treated or control group based on the paw scores on Day 16, when the first episode of inflammation was near the end. Relapse of arthritis was stimulated by intraperitoneal injection of 25μ1 of LPS, and a single dose of 20 mg of 2E7 or isotype control antibody was intraperitoneally injected to each mouse on the same day. The results demonstrate that treatment with 2E7 effectively prevented the increase in clinical paw scores, as compared to the control group. n=4.

FIG. 11 demonstrates that 2E7 and TNF-α blocker (etanercept) combined therapy is more efficacious than 2E7 or TNF-α blocker mono-therapy. CAIA mice were treated with a single dose of IP injection of 2E7, etanercept, control antibody, 2E7+etanercept or control antibody+etanercept at doses of 2E7 and etanercept as shown in the figure.

FIG. 12 shows that 2E7 did not suppress arthritis in NOD2 knockout CAIA mouse models. Injection of 2E7 did not significantly suppress the progression of arthritis in NOD2 knockout CAIA mouse models, which indicates that 2E7 suppresses rheumatoid arthritis by mainly, if not entirely, blocking the NOD2 signaling pathway.

FIG. 13 shows the effect of 2E7 in the treatment of multiple sclerosis (MS) is mouse experimental autoimmune encephalomyelitis (EAE) model, the most commonly used mouse model for the study of human MS. (A) shows that paralysis of the limbs and tail were suppressed after receiving three doses of 2E7. In other words, 2E7 treatment suppresses clinical symptoms of the disease, such as paralysis. (B) shows that the loss of body weight was significantly reduced after receiving three doses of 2E7. In other words, 2E7 treatment prevents the severe body weight loss in diseased mice. Mice treated with the isotype control antibody were used as the negative control, while mice receiving FTY720, a widely used small molecule drug, were used as the positive control in this example. *p<0.05; n.s.: not significant, n=12.

FIG. 14 shows that 2E7 significantly reduced the level of pro-inflammatory cytokines including SIL-1RI, sIL-6R, G-CSF and sVEGFR3, as compared to the control. Luminex assay was performed on the sera from the CAIA mice treated with control or 2E7 antibody. Significant reductions in G-CSF, sIL-1RI, sIL-6R and sVEGFR3 levels in sera were observed in 2E7 treated mice (gray) compared with control mice (black), n=8, *p<0.05, **p<0.01.

FIG. 15 shows that 2E7 significantly reduced T cell proliferation in CD4⁺ T-cells and CD8⁺ T-cells. Results are from flow cytometry analysis of splenocytes of CAIA mice treated with of 2E7 and control antibody. n=4.

### DETAILED DESCRIPTION OF THE PRESENT INVENTION

Muramyl peptides are fragments of peptidoglycan from the cell walls of bacteria. Because of their unique chemistry, the immune system recognizes muramyl peptides as products of bacteria, and it responds to muramyl peptides by becoming activated to resist infection. A key mechanism of the resistance to infection is activation of macrophages. Macrophage activation results in increased production of microbicidal oxygen radicals like superoxide and peroxide, and in increased secretion of inflammatory cytokines like interleukin-1-beta and tumor necrosis factor-alpha. These cytokines in turn activate neutrophils, B lymphocytes, and T lymphocytes to induce immunological responses. Some of these immunological responses result in immune-mediated conditions or diseases.

Accordingly, the inventors of the present disclosure envisage that antibodies that are directed to the various forms of muramyl peptides would be advantageous. Thus, the present invention provides for an antibody that is capable of binding to a muramyl peptide. As the binding of the antibody of the present disclosure to muramyl peptide blocks the biological activities of muramyl peptide (for example, activation of pro-inflammatory responses), the antibody of the present disclosure may be used for prevention and treatment of immune-mediated conditions or diseases. Accordingly, in a first aspect, there is provided a method of prophylactically or therapeutically treating an autoimmune or inflammatory disease comprising administering an isolated antibody or an antigen-binding fragment thereof, wherein the isolated antibody or antigen-binding fragment thereof is capable of binding to a muramyl peptide, or a derivative or an analog or a salt thereof.

As used herein the term “treatment”, or grammatical variants thereof, refers to any and all uses which remedy a disease state or symptoms, prevent the establishment of disease, or otherwise prevent, hinder, retard, or reverse the progression of disease or other undesirable symptoms in any way whatsoever. Treatment may be effected prophylactically (prior to the onset of the disease) or therapeutically (following diagnosis of the disease).

The term “antibody” as used herein refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to initiate an effector activity, or to bind to an antigen in a sample, for example in a solution, in a cell or tissue).

An “isolated antibody,” as used herein, refers to an antibody which is substantially free of other antibodies having different antigenic specificities (for instance an isolated antibody that specifically binds to a muramyl peptide is substantially free of antibodies that specifically bind antigens other than a muramyl peptide). An isolated antibody that specifically binds to an epitope, isoform or variant of a muramyl peptide may, however, have cross-reactivity to other related antigens, for instance from other bacterial species (such as a muramyl peptide species homologs). Moreover, an isolated antibody may be substantially free of other cellular material and/or chemicals.

In one embodiment, the antigen is a muramyl peptide. Muramyl peptide is a hallmark of bacterial peptidoglycan that is formed by parallel arrays of long sugar chains cross-linked by regularly spaced short peptide bridges. Accordingly, as used herein, the term “muramyl peptide” refers to peptidoglycan fragments or subunits containing at least one muramyl residue linked to a peptide. In one embodiment, the muramyl peptide, or a derivative or an analog or a salt thereof, is part of a peptidoglycan or fragment thereof. Thus, in one embodiment, the muramyl peptide may comprise muramic acid and an amino acid.

The glycan chain of peptidoglycan is composed of alternating residues of N-acetylglucosamine and N-acetylmuramic acid linked by β-1,4-glycosidic bonds. Muramic acid has a lactyl side chain on carbon 3 through which the glycan chains are covalently linked to the peptides. Muramic acid residues in different bacterial species may have different side chains at different carbon atoms. For example, many species have an N-acetyl group at carbon 2 and some species do not; and some species have a 1-6-anhydro linkage. Thus, in one embodiment, the muramic acid of the present disclosure may comprise an N-acetyl group. In another embodiment, the muramic acid does not comprise an N-acetyl group.

The amino acid in the muramyl peptide of the present disclosure may include, but is not limited to any amino acids found in bacterial peptidoglycan. In one embodiment, the amino acid in the muramyl peptide of the present disclosure may include proteinogenic amino acid and/or non-proteinogenic amino acid. In one embodiment, the proteinogenic amino acid may include, but is not limited to arginine, histidine, lysine, aspartic acid, glutamic acid, serine, threonine, asparagine, glutamine, cysteine, glycine, proline, alanine, valine, isoleucine, leucine, methionine, phenylalanine, tyrosine, and tryptophan; the non-proteinogenic amino acid may include, but is not limited to homoserine, lanthionine, ornithine, iso-glutamine, diaminobutyric acid, a-amino-n-butyric acid, norvaline, valine, norleucine, alloisoleucine, t-leucine, α-amino-n-heptanoic acid, pipecolic acid, α,β-diaminopropionic acid, α,γ-diaminobutyric acid, allothreonine, homocysteine, β-alanine, β-amino-n-butyric acid, β-aminoisobutyric acid, γ-aminobutyric acid, α-aminoisobutyric acid, isovaline, sarcosine, N-ethyl glycine, N-isopropyl glycine, N-methyl alanine, N-ethyl alanine, N-methyl β-alanine, N-ethyl β-alanine, isoserine, α-hydroxy-γ-aminobutyric acid and mesodiaminopimelic acid. In one embodiment, the amino acid in the muramyl peptide of the present disclosure may include, but is not limited to alanine, isoglutamine, glutamic acid, diaminobutyric acid, mesodiaminopimelic acid, glycine, homoserine, lanthionine, lysine, ornithine, serine and a salt thereof. In one embodiment, the amino acid in the muramyl peptide of the present disclosure may include, but is not limited to alanine, isoglutamine, glutamic acid, and a salt thereof.

In one embodiment, any of the amino acids may be provided as either an L-amino acid or a D-amino acid. As used herein, “L-amino acids” and “D-amino acids” refer to the two isomers that can occur in every amino acid. “L-amino acids” refer to the amino acid isomer which are manufactured in cells and incorporated into proteins. “D-amino acids” refers to isomeric modification to the amino acid as described herein. In one embodiment, the amino acid in the muramyl peptide of the present disclosure may be provided in an L-D, L-D-L-D, L-D-L-D-L-D, L-D-L-D-L-D-L-D, L-D-L-D-L-D-L-D-L-D, or L-D-L-D-L-D-L-D-L-D-L-D amino acid formation.

In one embodiment, the muramyl peptide may comprise or consist of two amino acids and may be referred to “muramyl dipeptide”. Thus, muramyl dipeptide comprises a muramic acid and a dipeptide. The term “dipeptide” as used herein refers to a string of amino acid that consists of two amino acids covalently linked to one another. As used herein, the string of amino acids covalently linked to the muramyl acid may be called peptide bridges. When the muramyl peptide is a muramyl dipeptide, the amino acid may comprise or consist of L-alanine or a salt thereof, and D-isoglutamine or a salt thereof. In another embodiment, the amino acid may comprise or consist of L-alanine or a salt thereof, and D-glutamic acid or a salt thereof.

In another embodiment, the muramyl peptide may comprise or consist of three amino acids and may be referred to as “muramyl tripeptides”. In one embodiment of a muramyl tripeptide, the amino acid may comprise or consist of L-alanine or a salt thereof, D-isoglutamine or D-glutamate or a salt thereof, and L-lysine or mesodiaminopimelic acid or a salt thereof.

In another embodiment, the muramyl peptide may comprise or consist of four amino acids and may be referred to as “muramyl tetrapeptides”. In one embodiment, the first amino acid of the peptide chain of a muramyl peptide may be an L-alanine. The second in the sequence may be a D-amino acid, for example D-isoglutamine or D-glutamate. The third amino acid may be linked to the γ-carboxyl group of the second amino acid instead of the conventional α-carboxyl group found in proteins. Thus, the third amino acid may be an L-diamino acid such as L-lysine or mesodiaminopimelic acid (mDAP). The fourth amino acid may be a D-alanine. Thus, the peptide chain of the muramyl peptide of the present disclosure may be L-D-L-D sequence, unlike the all L-amino acid sequence of proteins.

In yet another embodiment, the muramyl peptide may comprise or consist of five, six, seven, eight, nine, ten, 11, 12, 13, 14, 15 or more amino acids. As known in the art, the muramyl peptide may comprise or consist of a linear or a branched peptide. For example, a tetrapeptide from parallel peptidoglycan chains may be covalently linked between the D-alanine at the end of one peptide and the mDAP from the other peptide, which extends the length of peptide to seven with a one-amino acid (D-ala) branch. In some embodiments, there may be provided a 5-glycine linker joining the D-alanine and mDAP extending the length of the peptide to 11 with a D-alanine branch.

Advantageously, the antibody of the present disclosure may be capable of binding to the muramyl peptide as a whole; for example, the antibodies may be capable of binding to the muramic acid, the amino acid or the dipeptide as a group. Additionally, in contrast to the common general knowledge of N-acetyl group of muramic acid being an important antigenic determinant, the antibody of the present disclosure may bind to a muramyl peptide with or without N-acetyl group. In one embodiment, the antibody of the present disclosure may bind to a muramyl peptide and not to any of its subcomponents such as alanine, glutamic acid, iso-glutamic acid, muramic acid, or N-acetyl muramic acid. Without wishing to be bound by theory, it is envisaged that the ability of the antibody of the present disclosure to only bind muramyl peptides, and not to its subcomponents, provides the antibody its high specificity for muramyl peptides and peptidoglycan. Thus, in one embodiment, the antibody of the present disclosure may be capable of binding to the muramyl peptide, or a derivative or an analog or a salt thereof, that includes, but is not limited to N-acetylmuramyl-L-alanyl-D-isoglutamine, muramyl-L-alanyl-D-isoglutamine, N-acetylmuramyl-L-alanyl-D-glutamate, muramyl-L-alanyl-D-glutamate and the like.

An example of the antibody of the present disclosure includes 2E7, which comprises a heavy chain encoded by the nucleotide sequence of ATGCTGGTGGAGTCTGGGGGAGGCTTGGTGCAACCTGGAGGATCCATGAAACTCT CCTGTATAGTCTCGGGATTTACTTTCAGTTATTATTGGATGTCTTGGGTCCGCCAGT CTCCAGAGAAGGGGTTTGAGTGGGTTGCTGAAATCAGATTGAAATCTGAGAATTA TGCAACAAATTATACGGAGTCTGTGAAAGGGAAGTTCACCATCTCAAGAGATGAT TCCAAAAGTCGTCTCTACCTGCAAATGAACAGCTTAGGAGCTGAGGACACTGGAA TTTATTACTGTCTAACTGGTTATGCCTGGTTTGCTTATTGGGGCCAAGGGACTCTAG TCACTGTCTCTGCAGCCAAAACGACACCCCCATCTGTCTATCCACTGGCCCCTGGA TCTGCTGCCCAAACTAACTCCATGGTGACCCTGGGATGCCTGGTCAAGGGCTATTT CCCTGAGCCAGTGACAGTGACCTGGAACTCTGGATCCCTGTCCAGCGGTGTGCAC ACCTTCCCAGCTGTCCTGCAGTCTGACCTCTACACTCTGAGCAGCTCAGTGACTGT CCCCTCCAGCACCTGGCCCAGCGAGACCGTCACCTGCAACGTTGCCCACCCGGCC AGCAGCACCAAG (SEQ ID NO: 1).

In one embodiment, the isolated antibody or an antigen-binding fragment may comprise or consist of a light chain encoded by the nucleotide sequence of GACGTCCAGATGATCCAGTCTCCAAAGCGCCTAATCTATCTGGTGTCTAAACTGGA CTCTGGAGTCCCTGACAGGTTCACTGGCAGTGGATCAGGAACAGATTTTACACTG AAAATCAGCAGAGTGGAGGCTGAGGATTTGGGAGTTTATTACTGCGTGCAACATA CACATTTTCCCACGTTCGGAGGGGGGACCAAGCTGGAAATAAAACGGGCTGATGC TGCACCAACTGTATCCATCTTCCCACCATCCAGTGAGCAGTTAACATCTGGAGGTG CCTCAGTCGTGTGCTTCTTGAACAACTTCTACCCCAAAGACATCAATGTCAAGTGG AAGATTGATGGCAGTGAACGACAAAATGGCGTCCTGAACAGTTGGACTGATCAGG ACAGCAAAGACAGCACCTACAGCATGAGCAGCACCCTCACGTTGACCAAGGACG AGTATGAACGACATAACAGCTATACCTGTGAGGCCACTCACAAGACATCAACTTC ACCCATTGTCAAGAGCTTCAACAGGAATGAGTGT (SEQ ID NO: 2).

In one embodiment, the isolated antibody or an antigen-binding fragment thereof may comprise or consist of a heavy chain encoded by the nucleotide sequence of SEQ ID NO: 1 and a light chain encoded by the nucleotide sequence of SEQ ID NO: 2.

In one embodiment, the isolated antibody or an antigen-binding fragment thereof may comprise or consist of a heavy chain variable domain comprising the amino acid sequence as set forth in MLVESGGGLVQPGGSMKLSCIVSGFTFSYYWMSWVRQSPEKGFEWVAEIRLKSENYA TNYTESVKGKFTISRDDSKSRLYLQMNSLGAEDTGIYYCLTGYAWFAYWGQGTLVTV SAAKTTPPSVYPLAPGSAAQTNSMVTLGCLVKGYFPEPVTVTWNSGSLSSGVHTFPA VLQSDLYTLSSSVTVPSSTWPSETVTCNVAHPASSTK (SEQ ID NO: 3), or a variant thereof. The variant may comprise an amino acid sequence having at least 70%, at least 75%, at least 80%, at least 85%, at least 90%, at least 95%, at least 96%, at least 97%, at least 98%, or at least 99% identity with the amino acid sequence as set forth in SEQ ID NO: 3, whilst still retaining at least a substantial proportion (at least about 50%, 60%, 70%, 80%, 90%, 95% or more) of the affinity/avidity and/or the specificity/selectivity of the parent antibody and in some cases such an antibody may be associated with greater affinity, selectivity and/or specificity than the parent antibody.

In one embodiment, the isolated antibody or an antigen-binding fragment thereof may comprise or consist of a light chain variable domain comprising the amino acid sequence as set forth in DVQMIQSPKRLIYLVSKLDSGVPDRFTGSGSGTDFTLKISRVEAEDLGVYYCVQHTHF PTFGGGTKLEIKRADAAPTVSIFPPSSEQLTSGGASVVCFLNNFYPKDINVKWKIDGSE RQNGVLNSWTDQDSKDSTYSMSSTLTLTKDEYERHNSYTCEATHKTSTSPIVKSFNR NEC (SEQ ID NO: 4), or a variant thereof. The variant may comprise an amino acid sequence having at least 70%, at least 75%, at least 80%, at least 85%, at least 90%, at least 95%, at least 96%, at least 97%, at least 98%, or at least 99% identity with the amino acid sequence as set forth in SEQ ID NO: 4, whilst still retaining at least a substantial proportion (at least about 50%, 60%, 70%, 80%, 90%, 95% or more) of the affinity/avidity and/or the specificity/selectivity of the parent antibody and in some cases such an antibody may be associated with greater affinity, selectivity and/or specificity than the parent antibody.

In one embodiment, the isolated antibody or an antigen-binding fragment thereof as described herein, may comprise or consist of a heavy chain variable domain comprising the amino acid sequence as set forth in SEQ ID NO: 3, or a variant thereof, and a light chain variable domain comprising the amino acid sequence as set forth in SEQ ID NO: 4, or a variant thereof. The variant may comprise an amino acid sequence having at least 70%, at least 75%, at least 80%, at least 85%, at least 90%, at least 95%, at least 96%, at least 97%, at least 98%, or at least 99% identity with the amino acid sequence as set forth in SEQ ID NO: 3 or SEQ ID NO: 4, whilst still retaining at least a substantial proportion (at least about 50%, 60%, 70%, 80%, 90%, 95% or more) of the affinity/avidity and/or the specificity/selectivity of the parent antibody and in some cases such an antibody may be associated with greater affinity, selectivity and/or specificity than the parent antibody.

“Sequence Identity” as used herein refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods. Methods to determine sequence identity are codified in publicly available computer programs that determine sequence identity between given sequences. Examples of such programs include, but are not limited to, BLASTP, BLASTN and FASTA. The BLASTX program is publicly available from NCBI and other sources. These programs optimally align sequences using default gap weights in order to produce the highest level of sequence identity between the given and reference sequences.

The term antibody also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments). Accordingly, in one embodiment, the isolated antibody or an antigen-binding fragment thereof of the present disclosure may be selected from the group consisting of a humanized antibody and a chimeric antibody.

The term “monoclonal antibody” as used herein refers to a preparation of antibody molecules of single molecular composition. A monoclonal antibody composition displays a single binding specificity and affinity for a particular epitope. The monoclonal antibodies may be generated by a hybridoma which includes a B cell obtained from a transgenic nonhuman animal, such as a transgenic mouse, having a genome comprising a heavy chain transgene and a light chain transgene which encodes the antibody of the present disclosure, fused to an immortalized cell. Thus, in one embodiment, the isolated antibody or an antigen-binding fragment thereof may be a monoclonal antibody.

An antibody of the present disclosure may possess any isotype. Accordingly, in one embodiment, the isolated antibody may be of isotype IgG1, IgG2, IgG3, IgG4, IgD, IgA, IgE, or IgM. In yet another embodiment, the isolated antibody or an antigen-binding fragment thereof may be a monoclonal antibody and may be of the subtype IgG1.

An antibody of the present disclosure may also include fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the V_{L}, V_{H}, C_{L} and C_{H}1 domains, or a monovalent antibody; (ii) F(ab′)₂ fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment, which consists essentially of a V_{H} domain and also called domain antibodies; (vi) camelid or nanobodies and (vii) an isolated complementarity determining region (CDR). Accordingly, in one embodiment, the antigen-binding fragment may be selected from the group consisting of Fab, Fab′, (Fab′)₂, Fv, sFV, and scFv.

Advantageously, the antibody or antigen-binding fragment may bind to the muramyl peptide, or a derivative or an analog or a salt thereof, with a Kd value significantly less than values known in the art. As used herein, the term “Kd” (M) refers to the dissociation equilibrium constant of a particular antibody-antigen interaction. In one embodiment, the Kd may be selected from the group consisting of less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, and less than about 10 pM. For example, 2E7 mAb, which is one example of the antibody of the present disclosure, has been shown to have picomolar affinity. This is in contrast to other monoclonal antibody known in the art (i.e. mAb2-4), wherein inhibition assays using mAb2-4 showed that 50% inhibition of mAb2-4 binding to peptidoglycan by N-acetylmuramyl-L-alanyl-D-isoglutamine occurred only at concentrations higher than 1 mg/ml. Thus, the antibody of the present disclosure advantageously has higher binding affinity as compared to other known monoclonal antibody.

In a second aspect, there is provided an isolated antibody or an antigen-binding fragment thereof as described herein, for use in the prophylactic or therapeutic treatment of an autoimmune or inflammatory disease.

In a third aspect, there is provided use of an isolated antibody or an antigen-binding fragment thereof as described herein in the manufacture of a medicament for prophylactically or therapeutically treating an autoimmune or inflammatory disease.

The autoimmune or inflammatory disease that may be treated using the antibody of the present disclosure may be selected from the group consisting of sepsis, septic shock, Crohn's disease, rheumatoid arthritis, asthma, allergy, atopic disorders, multiple sclerosis, pertussis, gonorrhea, inflammatory bowel disease, and antibiotic-associated disorder. The treatment may comprise administering to a subject an antibody as described herein.

In one embodiment, the autoimmune or inflammatory disease is rheumatoid arthritis.

In one embodiment, the autoimmune or inflammatory disease is multiple sclerosis.

It may be advantageous in some cases for the antibody of the present disclosure to be administered with one or more other therapeutic agents to achieve better results of treatment and/or to reduce potential side effects. Examples of potential side effects include but are not limited to cancer and infection, such as bacterial infection and fungal infection. In some examples, the bacterial infection is caused by legionella or listeria. For example, a known side effect of TNF-α blocker is an increase in susceptibility of the patient to Listeria bacterial infection.

Accordingly, in a fourth aspect, there is provided a composition comprising an isolated antibody or an antigen-binding fragment thereof as described herein, and one or more therapeutic agents. Optionally, the composition of the present disclosure may comprise one or more pharmaceutically acceptable carriers or excipients as described herein.

The one or more other therapeutic agents in the composition of the present disclosure may be therapeutic agents that are useful for the treatment of immune-mediated diseases. Accordingly, the one or more therapeutic agents may be selected from the group consisting of nonsteroidal anti-inflammatory drugs (NSAIDs), non-biologic and biologic disease-modifying anti-rheumatic drugs (DMARDs), immunosuppressants, and corticosteroids. Exemplary DMARDs include, but are not limited to, methotrexate, hydroxychloroquine, sulfasalazine, leflunomide, Tumor Necrosis Factor (TNF) inhibitors, T-cell costimulatory blocking agents, B cell depleting agents, Interleukin-6 (IL-6) inhibitors and Interleukin-1 (IL-1) receptor antagonists. Exemplary TNF inhibitors include, but are not limited to, etanercept, adalimumab, infliximab, certolizumab pegol and golimumab.

In one example, the administration of the composition as described herein refers to the administration of two or more therapeutic agents (inclusive of the isolated antibody or an antigen-binding fragment thereof as described herein) in combination. “In combination” means that the therapeutic agents are administered closely enough in time that the administration or presence of one alters the biological effects of the other. The therapeutic agents may be administered simultaneously (concurrently) or sequentially.

Simultaneous administration may be carried out, for example by mixing two or more agents prior to administration, or by administering the agents/therapies at the same point in time but at different anatomic sites or using different routes of administration, or administered at times sufficiently close that the results observed are indistinguishable from those achieved when the agents/therapies are administered at the same point in time.

Sequential administration may be carried out by administering the agents/therapies at different points in time, for example, administering an agent/therapy at some point in time prior to or after administration of one or more other agents/therapies, such that the administration of the agents/therapies in combination enhances the therapeutic effect of treatment. In some embodiments, the isolated antibody or an antigen-binding fragment thereof as described herein is administered at some point in time prior to the administration of another therapeutic agent as described herein. Alternatively, the isolated antibody or an antigen-binding fragment thereof as described herein is administered at some point in time after the administration of another therapeutic agent.

The compositions as described herein may be administered in a number of ways depending upon whether local or systemic treatment is desired. Administration may be topical, pulmonary (e.g., by inhalation or insufflation of powders or aerosols, including by nebulizer; intratracheal, intranasal, epidermal and transdermal) or systemic such as oral, and/or parenteral. Parenteral administration includes intravenous, intraarterial, subcutaneous, intraperitoneal or intramuscular injection or infusion; or intracranial, e.g., intrathecal or intraventricular, administration. In one example, the route of administration may be selected from the group consisting of systemic administration, oral administration, intravenous administration and parenteral administration

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Compositions as described herein include, but are not limited to, solutions, emulsions, and liposome-containing formulations. These compositions may be generated from a variety of components that include, but are not limited to, preformed liquids, self-emulsifying solids and self-emulsifying semisolids.

The formulations as described herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions as described herein may be formulated into any of many possible dosage forms including, but not limited to tablets, capsules, liquid syrups, soft gels, suppositories, and enemas. The compositions as described herein may also be formulated as suspensions in aqueous, non-aqueous or mixed media. Aqueous suspensions may further contain substances that increase the viscosity of the suspension including, for example, sodium carboxymethyl cellulose, sorbitol and/or dextran. The suspension may also contain stabilizers.

In one embodiment, the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions as described herein may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the antibody(s) of the formulation.

The composition of the present disclosure may be useful in the treatment of immune-mediated diseases. Therefore, in a fifth aspect, there is provided a method of prophylactically or therapeutically treating an autoimmune or inflammatory disease as described herein comprising administering a composition of the present disclosure.

In a sixth aspect, there is provided a composition of the present disclosure for use in the prophylactic or therapeutic treatment of an autoimmune or inflammatory disease as described herein.

In a seventh aspect, there is provided use of the composition of the present disclosure in the manufacture of a medicament for prophylactically or therapeutically treating an autoimmune or inflammatory disease as described herein.

The composition as used herein may be provided in a therapeutically effective amount. The term “therapeutically effective amount” as used herein includes within its meaning a sufficient but non-toxic amount of the compound as described herein to provide the desired therapeutic effect. The exact amount required will vary from subject to subject depending on factors such as the species being treated, the age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of the composition, and can generally be estimated based on EC₅₀s found to be effective in in vitro and in vivo animal models or based on the examples described herein. In general, dosage is from 0.01 μs to 100 g/kg of body weight, and may be given once or more times daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g/kg of body weight, once or more times daily, to once every 2 years.

In one example, the dosage of the isolated antibody or antigen-binding fragment thereof is from 1 mg/kg to 1 g/kg of body weight, from 10 mg/kg to 1 g/kg of body weight, from 100 mg/kg to 1 g/kg of body weight, from 200 mg/kg to 1 g/kg of body weight, from 300 mg/kg to 1 g/kg of body weight, from 400 mg/kg to 1 g/kg of body weight, from 500 mg/kg to 1 g/kg of body weight, from 600 mg/kg to 1 g/kg of body weight, from 700 mg/kg to 1 g/kg of body weight, from 800 mg/kg to 1 g/kg of body weight, from 900 mg/kg to 1 g/kg of body weight, from 950 mg/kg to 1 g/kg of body weight, from 1 mg/kg to 950 mg/kg of body weight, from 1 mg/kg to 900 mg/kg of body weight, from 1 mg/kg to 800 mg/kg of body weight, from 1 mg/kg to 700 mg/kg of body weight, from 1 mg/kg to 600 mg/kg of body weight, from 1 mg/kg to 500 mg/kg of body weight, from 1 mg/kg to 400 mg/kg of body weight, from 1 mg/kg to 300 mg/kg of body weight, from 1 mg/kg to 200 mg/kg of body weight, or from 1 mg/kg to 100 mg/kg of body weight. Based on the dosages above, a person skilled in the art will be able to derive the dosages to be used in different animal species, including but not limited to human.

In one example, the dosage of the isolated antibody or antigen-binding fragment thereof is about 10 mg/kg of body weight.

In one example, the dosage of the isolated antibody or antigen-binding fragment thereof is about 160 mg/kg of body weight.

In one example, the compound may be administered in an amount of between any one of about 0.01 μg, 0.05 μg, 0.1 μg, 0.5 μg, 1 μg, 5 μg, 10 μg, 20 μg, 30 μs, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μs, 200 μg, 210 μg, 220 μg, 230 μs, 240 μg, 250 μs, 260 μs, 270 μg, 280 μs, 290 μg, 500 μs, 1 mg, 1.5 mg, 2 mg, 2.5 mg, 3 mg, 3.5 mg, 4 mg, 5 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg to any one of about 0.01 μg, 0.05 μg, 0.1 μs, 0.5 μg, 1 μg, 5 μg, 10 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 500 μg, 1 mg, 1.5 mg, 2 mg, 2.5 mg, 3 mg, 3.5 mg, 4 mg, 5 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg, 300 mg per kg of body weight of the subject. Based on the amounts above, a person skilled in the art will be able to derive the amounts to be used in different animal species, including but not limited to human.

In one example, the concentration of the administered compound is about 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 22.5 mg/ml, 25 mg/ml, 27.5 mg/ml, 30 mg/ml, 35 mg/ml, 40 mg/ml, 45 mg/ml, 50 mg/ml, 60 mg/ml, 70 mg/ml, 80 mg/ml, 90 mg/ml, 100 mg/ml, 150 mg/ml, 200 mg/ml, 250 mg/ml, 300 mg/ml, 350 mg/ml, 400 mg/ml, 450 mg/ml or about 500 mg/ml, or from about 0.1 mg/ml to about 500 mg/ml, from about 0.5 mg/ml to about 450 mg/ml, from about 1 mg/ml to about 400 mg/ml, from about 2 mg/ml to about 350 mg/ml, from about 3 mg/ml to about 300 mg/ml, from about 4 mg/ml to about 250 mg/ml, from about 5 mg/ml to about 200 mg/ml, from about 6 mg/ml to about 150 mg/ml, from about 7 mg/ml to about 100 mg/ml, from about 8 mg/ml to about 90 mg/ml, from about 9 mg/ml to about 80 mg/ml, from about 10 mg/ml to about 70 mg/ml, from about 11 mg/ml to about 60 mg/ml, from about 12 mg/ml to about 50 mg/ml, from about 12 mg/ml to about 45 mg/ml, from about 13 mg/ml to about 40 mg/ml, from about 14 mg/ml to about 35 mg/ml, from about 15 mg/ml to about 30 mg/ml, from about 16 mg/ml to about 27.5 mg/ml, from about 17 mg/ml to about 25 mg/ml, from about 18 mg/ml to about 22.5 mg/ml, or from about 19 mg/ml to about 20 mg/ml. In one example, the concentration of the administered compound is about 40 mg/ml. Based on the concentrations above, a person skilled in the art will be able to derive the concentrations to be used in different animal species, including but not limited to human.

In one example, the dosage of the other therapeutic agent to be administered with an isolated antibody or an antigen-binding fragment thereof of the present disclosure, is from 1 mg/kg to 1 g/kg of body weight, from 10 mg/kg to 1 g/kg of body weight, from 100 mg/kg to 1 g/kg of body weight, from 200 mg/kg to 1 g/kg of body weight, from 300 mg/kg to 1 g/kg of body weight, from 400 mg/kg to 1 g/kg of body weight, from 500 mg/kg to 1 g/kg of body weight, from 600 mg/kg to 1 g/kg of body weight, from 700 mg/kg to 1 g/kg of body weight, from 800 mg/kg to 1 g/kg of body weight, from 900 mg/kg to 1 g/kg of body weight, from 950 mg/kg to 1 g/kg of body weight, from 1 mg/kg to 950 mg/kg of body weight, from 1 mg/kg to 900 mg/kg of body weight, from 1 mg/kg to 800 mg/kg of body weight, from 1 mg/kg to 700 mg/kg of body weight, from 1 mg/kg to 600 mg/kg of body weight, from 1 mg/kg to 500 mg/kg of body weight, from 1 mg/kg to 400 mg/kg of body weight, from 1 mg/kg to 300 mg/kg of body weight, from 1 mg/kg to 200 mg/kg of body weight, or from 1 mg/kg to 100 mg/kg of body weight. Based on the dosages above, a person skilled in the art will be able to derive the dosages to be used in different animal species, including but not limited to human.

In one example, the dosage of the other therapeutic agent is about 5 mg/kg of body weight.

In one example, the other therapeutic agent that is to be administered with an isolated antibody or an antigen-binding fragment thereof of the present disclosure is etanercept, and the dosage of etanercept is 5 mg/kg of body weight.

As used herein, the term “about”, in the context of amounts or concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

In one embodiment, the subject that is treated with the antibody or composition of the present disclosure may be an animal, mammal, human, including, without limitation, animals classed as bovine, porcine, equine, canine, lupine, feline, murine, ovine, avian, piscine, caprine, corvine, acrine, or delphine. In one embodiment, the subject may be a human.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention will be further described in greater detail by reference to specific Examples, which should not be construed as in any way limiting the scope of the invention.

### Materials and Methods

Muramyl peptides (MPs) are related molecules sharing common structural moieties. In the present disclosure, two types of mouse monoclonal antibodies were developed with one recognizing a common structure and another specific for subtypes. To achieve this, the following muramyl dipeptides (MDPs) were used as antigens: N-acetylmuramyl-L-alanyl-D-isoglutamine, muramyl-L-alanyl-D-isoglutamine, N-acetylmuramyl-L-alanyl-D-glutamate, and muramyl-L-alanyl-D-glutamate. These MDPs were chemically synthesized or purified from partial HCl-hydrolysis products of N-acetylmuramyl-L-alanyl-D-isoglutamine as described previously (Xu et al., 2008, Bacterial peptidoglycan triggers Candida albicans hyphal growth by directly activating the adenylyl cyclase Cyr1p. Cell Host & Microbe 4, 1-12, the content of which is incorporated herewith by reference).

To enhance the antigenicity of the MDPs, these molecules were conjugated to the human serum albumin (HSA) using a linker molecule. The carboxylic acid moiety of MDPs was first coupled to N-Boc-ethylenediamine, and then the Boc protection group was removed and the resulting amine was linked to HSA with glutaraldehyde. Successful conjugation of MDPs to HSA was determined by mass spectrometry. The MDP-HSA conjugates were then used to immunize BALB/c mice. Serum antigen-specific titers of the immunized mice were examined by enzyme-linked immunosorbent assay (ELISA) against MDPs conjugated to ovalbumin (OVA) using the same linkage strategy as described above. Generation of hybridoma cell lines, screening of antibody-producing clones, preparation and purification of mAbs, and mAb isotyping were carried out by following standard protocols. Antigen specificity of a mAb was determined using competitive ELISA to test the ability of different MDPs and constituent moieties to inhibit the binding of a mAb to the MDP originally used as antigen for immunization.

### Example 1—Characterization of a mAb Against MDPs

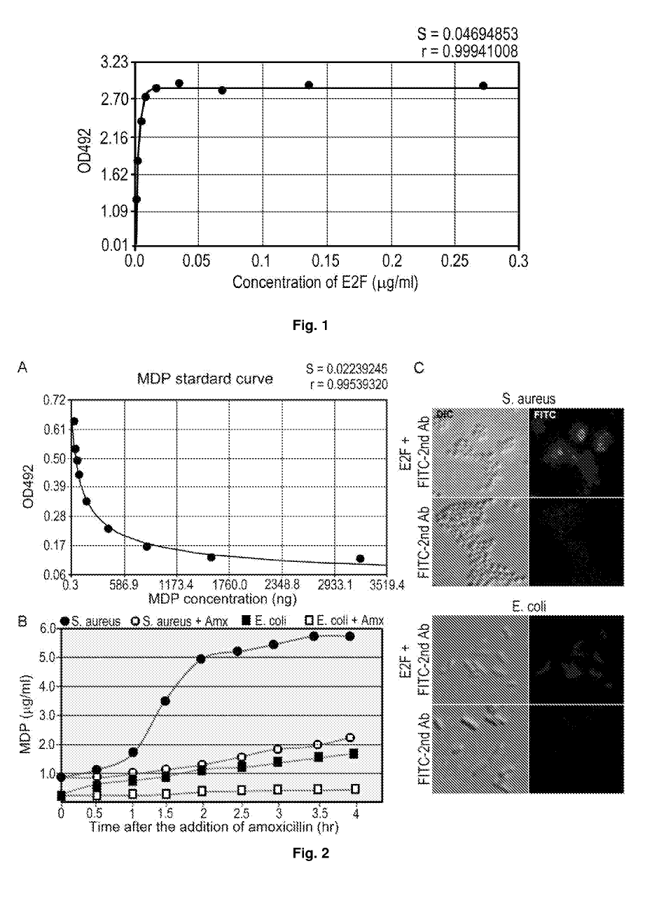

A mAb (2E7) was obtained from immunization of mice with N-acetylmuramyl-L-alanyl-D-isoglutamine. Antibody isotyping tests identified 2E7 as IgG₁ and the Kd of 2E7 for N-acetylmuramyl-L-alanyl-D-isoglutamine was calculated to be 8.7 pM (FIG. 1). By competitive ELISA, the binding of 2E7 to N-acetylmuramyl-L-alanyl-D-isoglutamine conjugated to OVA was found to be inhibited in a concentration dependent manner by muramyl-L-alanyl-D-isoglutamine, N-acetylmuramyl-L-alanyl-D-glutamate, and muramyl-L-alanyl-D-glutamate almost as effectively as N-acetylmuramyl-L-alanyl-D-isoglutamine, indicating that 2E7 recognizes a common epitope in the four MDPs. However, 2E7 did not exhibit detectable affinity to muramic acid, N-acetylmuramic acid, N-acetylglucosamine, alanine, D-isoglutamine, glutamate, glucose, or any or a mixture of the 20 common amino acids in proteins at concentrations 100 times higher than their normal concentrations in the blood. The data indicate that 2E7 specifically recognizes an epitope formed uniquely in a structural context common in the four MDPs. An IgG₁ mAb that specifically recognizes the glutaraldehyde linker used to couple MDP to HSA was also obtained in the screening of hybridoma clones. This antibody exhibited no detectable affinity to any of the above molecules except glutaraldehyde, providing an excellent negative control for experiments using 2E7.

### Example 2—Determination of the Amino Acid Sequences of the Variable Region of the Heavy and Light Chains of 2E7

Messenger RNAs were prepared from the hybridoma clone that produces 2E7 and then used as templates to produce complementary DNA. The DNA fragment encoding the variable region of the heavy and light chain respectively was amplified by polymerase chain reactions (PCR) using pairs of oligonucleotide primers (Table 1) specifically targeting conserved sequence motifs flanking the coding region for the variable region (method of which is described in Kettleborough et al., 1993, Optimisation of primers for cloning libraries of mouse immunoglobulin genes using the polymerase chain reaction. Eur J Immunol 23, 206-211 and Pope et al., 1996, Construction of use of antibody gene repertoires. In Antibody Engineering—A Practical Approach. Edited by McCafferty J. Hoogenboom H, and Chiswell D., the content of both are incorporated herewith by reference). The PCR products were purified, spliced into the pJET1.2/blunt vector (Fermentas International Inc, Canada) and transformed into Escherichia coli to obtain independent clones. Plasmids were isolated from multiple clones and the ones with an insert of the expected size were subjected to DNA sequence analysis. Five clones each for heavy and light chains were analyzed, which yielded identical sequences. The nucleotide sequences were then translated into amino acid sequences (Table 2). Their identity as the variable region of the heavy or light chain of mouse antibodies was confirmed by using the sequences to search the NCBI non-redundant protein sequence database. The 2E7 heavy chain sequence exhibited the highest identity of 75-90% to dozens of mouse antibodies over the same region, and the 2E7 light chain sequence exhibited identities up to 98%. No identical sequence in the database was found to either the heavy or light chain of 2E7.

TABLE 1
Oligonucleotide primers for PCR amplification of
the DNA coding sequence for the variable
region of the heavy and light chain of 2E7.

Heavy chain primers
Forward
1. 5′-ATGCTGGTGGAGTCTGGGGGA-3′ (SEQ ID NO: 5)
2. 5′-AAGCTGGTGGAATCTGGAGGA-3′ (SEQ ID NO: 6)
Reverse
5′-CTTGGTGCTGCTGGCCGGGTG-3′ (SEQ ID NO: 7)
Light chain primers
Forward
1. 5′-CCGTTTGATTTCCAGCTTGGTGCC-3′ (SEQ ID NO: 8)
2. 5′-CCGTTTCAGCTCCAGCTTGGTCCC-3′ (SEQ ID NO: 9)
Reverse
5′-GACATTGAGCTCACCCAGTCTCCA-3′ (SEQ ID NO: 10)

TABLE 2
Nucleotide and amino acid sequences of the
variable regions of 2E7

Heavy chain (208 amino acids) (SEQ ID NO: 3):
MLVESGGGLVQPGGSMKLSCIVSGFTFSYYWMSWVRQSPEKGFEWVAEIR
LKSENYATNYTESVKGKFTISRDDSKSRLYLQMNSLGAEDTGIYYCLTGY
AWFAYWGQGTLVTVSAAKTTPPSVYPLAPGSAAQTNSMVTLGCLVKGYFP
EPVTVTWNSGSLSSGVHTFPAVLQSDLYTLSSSVTVPSSTWPSETVTCNV
AHPASSTK
Nucleotide sequence (624 nucleotides)
(SEQ ID NO: 1):
ATGCTGGTGGAGTCTGGGGGAGGCTTGGTGCAACCTGGAGGATCCATGAA
ACTCTCCTGTATAGTCTCGGGATTTACTTTCAGTTATTATTGGATGTCTT
GGGTCCGCCAGTCTCCAGAGAAGGGGTTTGAGTGGGTTGCTGAAATCAGA
TTGAAATCTGAGAATTATGCAACAAATTATACGGAGTCTGTGAAAGGGAA
GTTCACCATCTCAAGAGATGATTCCAAAAGTCGTCTCTACCTGCAAATGA
ACAGCTTAGGAGCTGAGGACACTGGAATTTATTACTGTCTAACTGGTTAT
GCCTGGTTTGCTTATTGGGGCCAAGGGACTCTAGTCACTGTCTCTGCAGC
CAAAACGACACCCCCATCTGTCTATCCACTGGCCCCTGGATCTGCTGCCC
AAACTAACTCCATGGTGACCCTGGGATGCCTGGTCAAGGGCTATTTCCCT
GAGCCAGTGACAGTGACCTGGAACTCTGGATCCCTGTCCAGCGGTGTGCA
CACCTTCCCAGCTGTCCTGCAGTCTGACCTCTACACTCTGAGCAGCTCAG
TGACTGTCCCCTCCAGCACCTGGCCCAGCGAGACCGTCACCTGCAACGTT
GCCCACCCGGCCAGCAGCACCAAG
Light chain (177 amino acids) (SEQ ID NO: 4):
DVQMIQSPKRLIYLVSKLDSGVPDRFTGSGSGTDFTLKISRVEAEDLGVY
YCVQHTHFPTFGGGTKLEIKRADAAPTVSIFPPSSEQLTSGGASVVCFLN
NFYPKDINVKWKIDGSERQNGVLNSWTDQDSKDSTYSMSSTLTLTKDEYE
RHNSYTCEATHKTSTSPIVKSFNRNEC
Nucleotide sequence (531 nucleotides)
(SEQ ID NO: 2):
GACGTCCAGATGATCCAGTCTCCAAAGCGCCTAATCTATCTGGTGTCTAA
ACTGGACTCTGGAGTCCCTGACAGGTTCACTGGCAGTGGATCAGGAACAG
ATTTTACACTGAAAATCAGCAGAGTGGAGGCTGAGGATTTGGGAGTTTAT
TACTGCGTGCAACATACACATTTTCCCACGTTCGGAGGGGGGACCAAGCT
GGAAATAAAACGGGCTGATGCTGCACCAACTGTATCCATCTTCCCACCAT
CCAGTGAGCAGTTAACATCTGGAGGTGCCTCAGTCGTGTGCTTCTTGAAC
AACTTCTACCCCAAAGACATCAATGTCAAGTGGAAGATTGATGGCAGTGA
ACGACAAAATGGCGTCCTGAACAGTTGGACTGATCAGGACAGCAAAGACA
GCACCTACAGCATGAGCAGCACCCTCACGTTGACCAAGGACGAGTATGAA
CGACATAACAGCTATACCTGTGAGGCCACTCACAAGACATCAACTTCACC
CATTGTCAAGAGCTTCAACAGGAATGAGTGT

### Example 3-2E7 Detection of Bacterial Peptidoglycan in Culture Medium and on the Cell Surface

To demonstrate the utility of 2E7, the antibody was first tested on its ability to detect MPs that are normally present in bacterial cultures. It is well established that β-lactam antibiotics inhibit peptidoglycan polymerization by causing cells to accumulate and secrete MPs. β-lactam antibiotic amoxicillin, which is a drug commonly used in hospitals, were added to the culture and a significant increase in the substance that can inhibit 2E7 binding to N-acetylmuramyl-L-alanyl-D-isoglutamine was expected to increase. For this test, Gram-positive bacterium Staphylococcus aureus, which has a thick peptidoglycan layer, and Gram-negative Escherichia coli, which has a thin peptidoglycan layer, were selected and grown in the presence or absence of amoxicillin. The culture was grown to a density of OD₆₀₀=1.5 and then divided into two equal aliquots. To one aliquot, amoxicillin was added to a final concentration of 40 μg per ml, and to the other aliquot, no drug was added. Both cultures were allowed to continue to grow. Aliquots of cultures were collected at timed intervals and cells were removed by centrifugation. The amount of MPs in the supernatant was determined by competitive ELISA using 2E7. We found that the addition of amoxicillin caused a sharp increase in the culture medium of molecules that can effectively inhibit 2E7 binding to N-acetylmuramyl-L-alanyl-D-isoglutamine (FIG. 2A-B). In comparison, only a slow, gradual increase of such molecules was observed in untreated cultures. Furthermore, supernatant of the Staphylococcus aureus culture exhibited ˜15 times stronger inhibition than that of the Escherichia coli culture. As a negative control, none of the supernatants discernibly inhibited the binding of the control antibody against glutaraldehyde under otherwise identical conditions.

The ability of 2E7 in binding to peptidoglycan in the cell wall was also investigated. Both Staphylococcus aureus and Escherichia coli cells were first incubated with 2E7 and then with an anti-mouse IgG Ab conjugated with FITC. Fluorescence microscopy examination revealed strong staining of Staphylococcus aureus and weak staining of Escherichia coli cells (FIG. 2C), consistent with their respective level of peptidoglycan content in the cell wall. Incubation with the secondary FITC-Ab alone resulted in only faint non-specific staining. Furthermore, incubating 2E7 but not the control antibody with live Staphylococcus aureus or Escherichia coli caused significant cell aggregation (data not shown), indicating cross-linking of bacterial cells by 2E7. Taken together, the data indicate that the antibody of the present disclosure, 2E7, specifically recognizes peptidoglycan in bacterial cell wall and MPs released in bacterial cultures.

### Example 4—Influence of Amoxicillin on the MP Level in the Blood in Mice and Humans

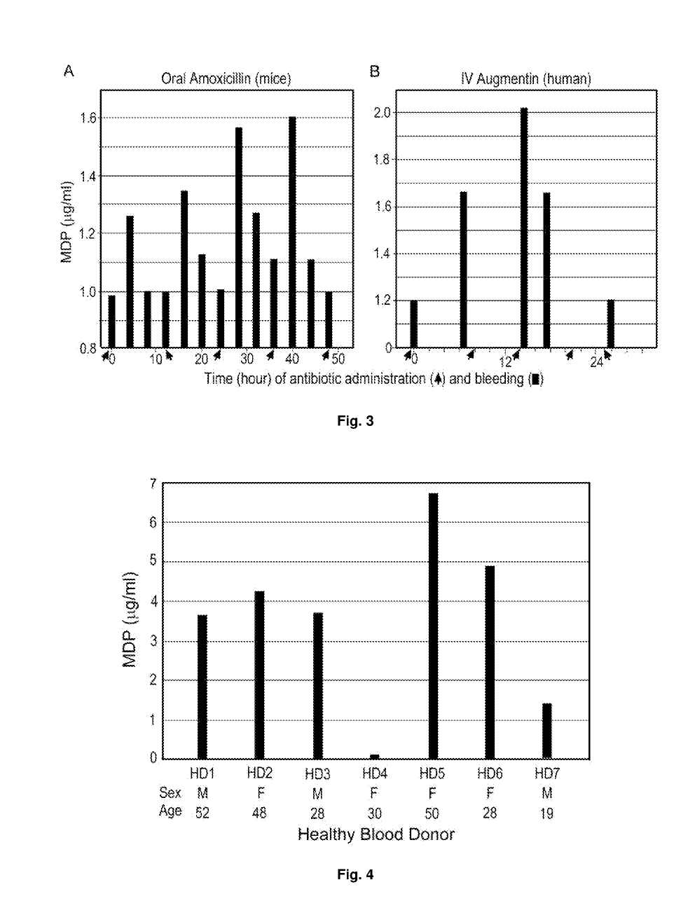

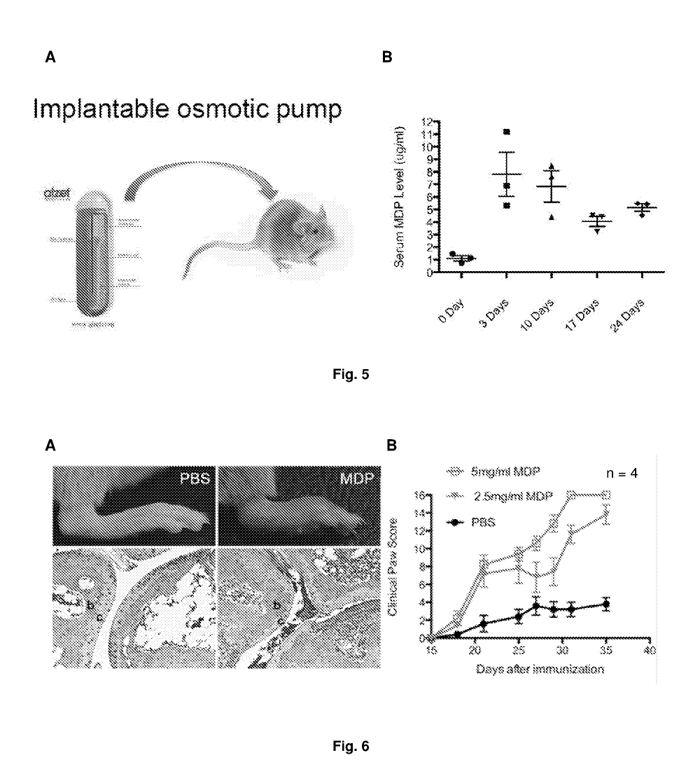

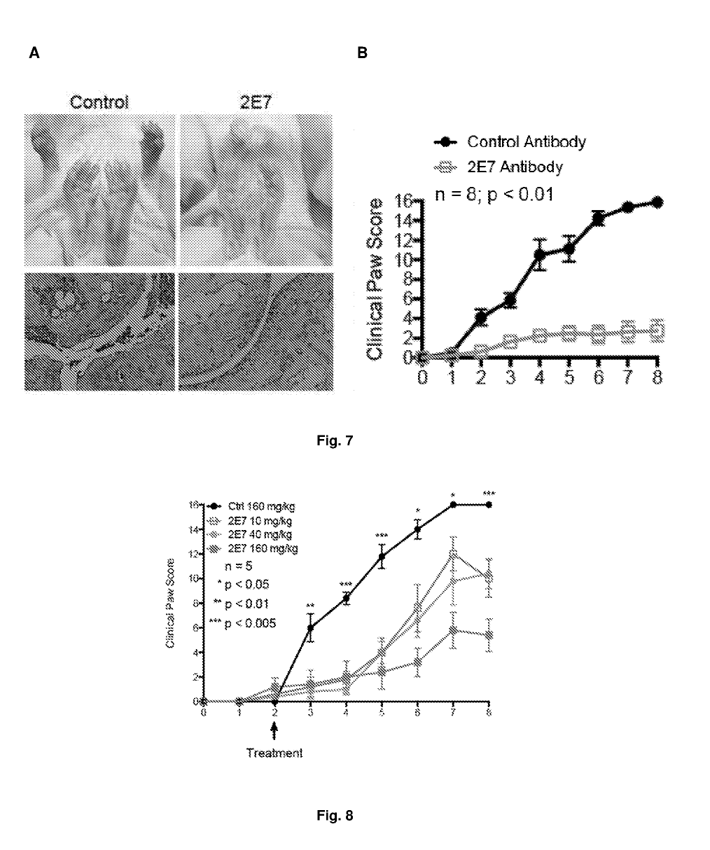

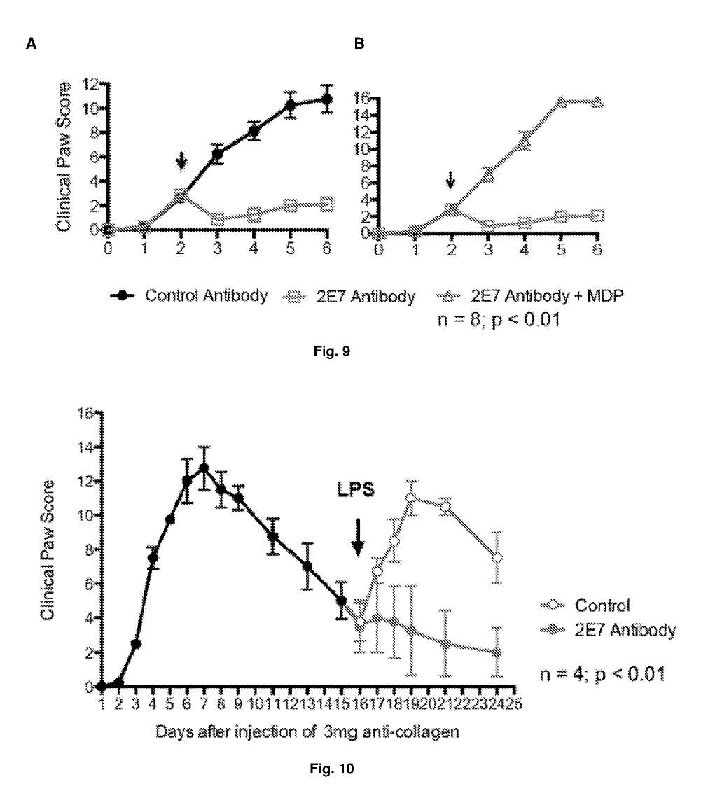

To determine whether β-lactam antibiotics treatment of mice would cause a sudden increase in the blood MP level, which can occur as a result of inhibition of peptidoglycan synthesis in bacteria of the mouse microbiota, mice were fed with amoxicillin (100 mg/kg) at 12-h intervals and three mice were sacrificed every 4 h to collect blood for a period of three days. The MP level in serum was determined by competitive ELISA using an example of the antibody of the present disclosure, i.e. 2E7. As shown in FIG. 3A, the serum of untreated mice (0 h) contains a level of MPs equivalent to ˜1 μg/ml of N-acetylmuramyl-L-alanyl-D-isoglutamine. Strikingly, a 20-60% increase in the blood MP level was observed at 4 h after each antibiotic feeding, although the level returned to the basal level during the next few hours until the next feeding.

At the same time, blood samples from an ICU patient who received multiple IV administration of Augmentin (amoxicillin+clavulanate, a β-lactamase inhibitor reducing amoxicillin degradation by bacterial (3-lactamase) was obtained serially at 0, 7.5 h, 14 h, 21 h, and 26 h. The blood sample taken before the antibiotic treatment contained an amount of MPs equivalent to ˜1.2 μg/ml of N-acetylmuramyl-L-alanyl-D-isoglutamine (FIG. 3B). As shown in FIG. 3B, the MP level increased to 1.66 and 2.1 μg/ml in samples taken at 6.5 and 13.5 h. In contrast, the MP level lowered to 1.68 and 1.2 μg/ml at 17 and 26 h. These results indicate that the use of β-lactam antibiotics indeed causes a significant increase of MPs in the blood. These results might explain the cause of some antibiotic-associated diseases such as diarrhea as a consequence of MP-induced excessive inflammation in the gut. The data also suggest the presence of regulatory mechanisms in mice and humans that can effectively restore the blood MPs to the homeostatic state level in response to MP fluctuations in the body.

### Example 5—Detection and Quantification of Blood MPs in Healthy People

It was hypothesized that due to genetic and/or environmental factors, MPs levels in individuals may vary. Since MPs have a diverse range of biological activities and have been linked to numerous diseases, the blood level of MPs may be used as a biomarker for assessment of risks of individuals for developing certain diseases. As a first step, the MP level in generally healthy people was determined. Blood samples and prepared serum from seven voluntary donors, four female and three male aged from 19 to 52 at 10:00 am of the same day (FIG. 4) were collected. Strikingly, the serum MP level was found to vary over a wide range. One serum (HD4) contained a barely detectable amount of MPs, while the highest MP concentration detected was 6.82 μg/ml (HD5). The levels of MPs in the rest of the serum samples ranged from 1.4 to 4.94 μg/ml. When blood samples were obtained again from HD4 and HD5 two weeks later, similar results were observed. From another independent group of four donors, a second serum sample was found to contain an undetectable level of MPs. The data indicate that the blood MP level indeed exhibits significant inter-individual variations. Incidentally, the two donors with the highest blood peptidoglycan levels, HD5 and HD6, particularly HD5, have chronic inflammation-related skin problems and often need to take antibiotics for extended periods of time for anti-inflammation purposes. The observed correlation between high blood MP levels and the skin problem suggests that the high MP level may be the cause of or contribute to chronic, low grade inflammation.

### Example 6—Prevention of Development, Treatment of Progression, and Prevention of Relapse of Rheumatoid Arthritis Using 2E7 mAb

Since MDPs are capable of inducing immunological responses, some of which result in immune-mediated conditions or diseases, it is hypothesized that the MDP antibody 2E7 can be used to treat immune-mediated diseases such as rheumatoid arthritis. To test this hypothesis, the effect of 2E7 was studied using the Collagen Antibody Induced Arthritis (CAIA) mouse model.

CAIA Mouse Model

CAIA was induced in 9-10 weeks old Balb/c mice by intravenous injection of 3 mg of Arthogen-CIA-5 cocktail from the tail vein on Day 0 and intraperitoneal injection of 25μ1 of LPS on Day 3.

Groups and Treatment

Preventive Study

For the study of 2E7 on the prevention of rheumatoid arthritis, mice were randomly assigned to 2E7 treatment group or control group according to body weight. A single dose of 20 mg of 2E7 or the isotype control antibody was intraperitoneally injected to each mouse 6 hours before induction of CAIA.

Therapeutic Study

To study the therapeutic effect of 2E7 on rheumatoid arthritis, mice were induced for CAIA as a single group on Day 0. On day 2, mice were assessed by paw score (Table 3) and assigned to groups in a manner such that different groups have similar average paw scores. The mice that were extremely sensitive to induction (score from a single paw=4) were excluded from the study. On Day 2, each mouse was given a single dose of 2E7 or the control antibody by intraperitoneal injection. On day 3, each mouse was given an intraperitoneal injection of 25 μl of LPS.

Relapse Study:

To study the effect of 2E7 in the prevention of the relapse of rheumatoid arthritis, mice were assigned to 2E7 treated or control group based on the paw scores on Day 16, when the first episode of inflammation was near the end. Relapse of arthritis was stimulated by intraperitoneal injection of 25μ1 of LPS, and a single dose of 20 mg of 2E7 or isotype control antibody was intraperitoneally injected to each mouse on the same day.

Measurement of Clinical Paw Score

Table 3 shows the criteria used to assess the clinical paw scores of the mice.

TABLE 3
Paw score criteria
The maximal score for one animal is 16 (adding the scores for all 4 paws)

| Paw score | Clinical observations |
|---|---|
| 0 | Normal |
| 1 | Mild redness, slight swelling of ankle or wrist |
| 2 | Moderate swelling of ankle or wrist |
| 3 | Severe swelling, including some digits, ankle and foot |
| 4 | Maximally inflamed |

### Example 7—Therapeutic Effect of 2E7 mAb at Different Doses in Mouse Collagen Antibody Induced Arthritis (CAIA) Model

To study the therapeutic effects of 2E7 at different doses, the same CAIA mouse model was used. Mice were induced for CAIA as a single group on Day 0. On day 2, mice were assessed by paw score (Table 3) and assigned to groups in a manner that different groups have similar average paw scores. The mice that were extremely sensitive to induction (score from a single paw=4) were excluded from the study. On Day 2, each mouse was given the control antibody or a single dose of 2E7 at 10 mg/kg, 40 mg/kg and 160 mg/kg of bodyweight by intraperitoneal injection. On day 3, each mouse was given an intraperitoneal injection of 25μ1 of LPS. The clinical paw scores of the mice were assessed according to the paw score criteria in Table 3. As shown in FIG. 8, all three doses of 2E7 showed clear and dose-dependent therapeutic effects. The fact that 2E7 is efficacious at 10 mg/kg indicates 2E7 has the potential to be developed into a potent therapeutics for RA.

### Example 8-2E7 mAb and TNF-α Blocker Combined Therapy for Treatment of Rheumatoid Arthritis

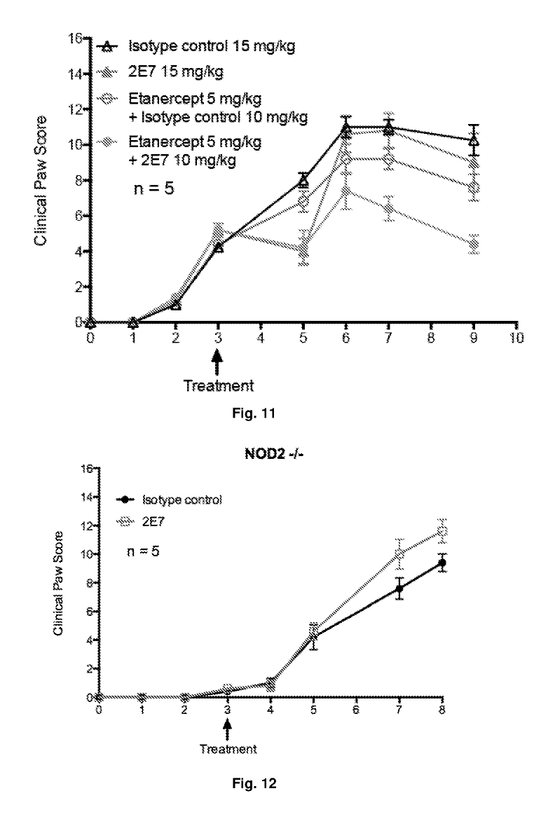

Since 2E7 was demonstrated to have therapeutic effect on rheumatoid arthritis, further studies were carried out to test the effect of combined therapy for the treatment if rheumatoid arthritis. The same CAIA mouse model was used. Mice were induced for CAIA as a single group on Day 0. On day 2, mice were assessed by paw score (Table 3) and assigned to groups in a manner that different groups have similar average paw scores. The mice that were extremely sensitive to induction (score from a single paw=4) were excluded from the study. On Day 2, each mouse was given (i) the control antibody at 15 mg/kg of body weight, (ii) a single dose of 2E7 at 15 mg/kg of body weight, (iii) a single dose of TNF-α blocker etanercept at 5 mg/kg of body weight plus the control antibody at 10 mg/kg of body weight, or (iv) a single dose of etanercept at 5 mg/kg of body weight plus the 2E7 at 10 mg/kg of body weight, by intraperitoneal injection. On day 3, each mouse was given an intraperitoneal injection of 25 μl of LPS. The clinical paw scores of the mice were assessed according to the paw score criteria in Table 3. As shown in FIG. 11, a combined therapy of 2E7 and etanercept was more efficacious than etanercept or 2E7 mono-therapy in mouse CAIA model. This result indicates that 2E7 can be developed as a companion therapy to current RA therapy to achieve better results. Advantageously, the synergistic effect given by 2E7 when used in combination of other currently available RA therapeutic agent(s) allows the concentrations of both 2E7 and the other agent(s) to be reduced significantly to achieve the same or better effect than either one used alone at much higher concentrations.

### Example 9—the Role of Nucleotide-Binding Oligomerization Domain-Containing Protein 2 (NOD2) in 2E7 mAb Treatment of Rheumatoid Arthritis

Since 2E7 is an MDP antibody, it is hypothesized that 2E7 exerts its effect on rheumatoid arthritis by neutralizing MDP-containing molecules in the circulation. NOD2 is an intracellular pattern recognition receptor, which is similar in structure to resistant proteins of plants and recognizes molecules containing the specific structure of MDP. If 2E7 exerts its effect on rheumatoid arthritis by neutralizing MDP-containing molecules in the circulation, its efficacy would be abolished in mice without NOD2 (NOD2−/−). To test this hypothesis, Nod2 knock-out mice in C57B/L6 background were induced for CAIA as a single group on Day 0, by intravenous injection of 5 mg of Arthogen-CIA-5 cocktail from the tail vein. On day 2, mice were assessed by paw score (Table 3) and assigned to groups in a manner that different groups have similar average paw scores. The mice that were extremely sensitive to induction (score from a single paw=4) were excluded from the study. On Day 2, each mouse was given a single dose of 2E7 or the control antibody by intraperitoneal injection. On day 3, each mouse was given an intraperitoneal injection of 50 μl of LPS. Higher amount of Arthogen-CIA-5 cocktail and LPS were used since mice in C57B/L6 background are more resistant to the CAIA induction than mice in Balb/c background. As shown in FIG. 12, injection of 2E7 was not able to suppress disease progression. This data show that 2E7 treats RA by mainly, if not entirely, blocking the NOD2 signaling pathway.

### Example 10—Therapeutic Effect of 2E7 mAb on Multiple Sclerosis (MS) in Mouse Experimental Autoimmune Encephalomyelitis (EAE) Model

Multiple sclerosis is the prototypical inflammatory demyelinating disease of the central nervous system caused by autoimmunity. It is estimated to affect up to two million people worldwide. As 2E7 has shown efficacy in mouse model for rheumatoid arthritis, an autoimmune disease in joints, it is hypothesized that it would be able to suppress multiple sclerosis as well. Experimental Autoimmune Encephalomyelitis (EAE) mouse model was used to test this hypothesis.

EAE Induction

EAE induction was carried out in 42 female C57BL/6 mice (Taconic Farms, 9 weeks old). The induction was carried out according to the following schedule:

Day 0, Hour 0—Immunization with MOG₃₅₋₅₅/CFA
Day 0, Hour 2—Injection of pertussis toxin
Day 1, Hour 0—2nd injection of pertussis toxin (24 hours after initial immunization)

Mice were injected subcutaneously at two sites in the back with the emulsion component (containing MOG₃₅₋₅₅) of Hooke Kit™ M OG₃₅₋₅₅/CFA Emulsion PTX, catalog number EK-2110 (Hooke Laboratories, Lawrence Mass.). One site of injection was in the area of upper back, approximately 1 cm caudal of the neckline. The second site was in the area of lower back, approximately 2 cm cranial of the base of the tail. The injection volume was 0.1 mL at each site. Within 2 hours of the injection of emulsion, and then again 24 hours after the injection of emulsion, the pertussis toxin component of the kit was administered intraperitoneally. The volume of each injection was 0.1 mL. To optimize disease severity for this particular study, the pertussis toxin from Hooke Kit™ MOG₃₅₋₅₅/CFA Emulsion PTX, catalog number EK-2110 was diluted with PBS to achieve 133 ng/dose for the first injection and 144 ng/dose for the second injection.

Groups and Treatment

Before treatment, all mice were initially considered as a single group. After daily scoring, each mouse with newly developed clinical signs of EAE was assigned to one of the experimental groups 1 to 3 in a balanced manner to achieve groups with similar time to EAE onset and similar onset scores. Mice which developed very late EAE onset or which developed unusual signs of EAE such as head tilting were not assigned to any treatment group. Different treatment regimens as set out in Table 4 were administered to mice in groups 1 to 3.

TABLE 4
Treatment regimens for mice in Groups 1 to 3

| | # | | | | | | |
|---|---|---|---|---|---|---|---|
| Group | mice | Treatment | Dose | Route | Frequency | Volume | Purpose |
| 1 | 12 | Isotype | 150 mg/kg | i.p. | 1^{st}, 4^{th} and 7^{th} | 10 mL/kg | Negative |
| | | control | | | day of disease | | control |
| 2 | 12 | FTY720 | 3 mg/kg | i.p. | Daily | 10 mL/kg | Positive |
| | | | | | | | control |
| 3 | 12 | 2E7 | 150 mg/kg | i.p. | 1^{st}, 4^{th} and 7^{th} | 10 mL/kg | Test |
| | | | | | day of disease | | |

Treatments of the newly-assigned mice were initiated on the day of the assignment, which was the first day of clinical disease. Mice in Group 2 were dosed daily. Mice in Groups 1 and 3 were treated on the first day of the disease and again on the fourth and seventh day of disease. Treatments were done at the same time (+/−1 hour) of each day.

Scoring and Readout

EAE scores were measured daily from Day 7 after immunization until the end of the study according to the criteria set out in Table 5. Body weights of the mice were measured three times a week (Monday, Wednesday and Friday), starting from Day −1 and until the end of the study. The last day of scoring was 15 days after assignemnt for each mouse. Scoring was performed blind, by a person unaware of the treatment administered and the previous scores for each mouse.

TABLE 5
EAE Scoring Criteria

| Score | Clinical observations |
|---|---|
| 0 | No obvious changes in motor functions of the mouse in comparison |
| | to non-immunized mice. |
| | When picked up by the tail, the tail has tension and is erect. Hind |
| | legs are usually spread apart. When the mouse is walking, there is |
| | no gait or head tilting. |
| 1 | Limp tail. |
| | When the mouse is picked up by the tail, instead of being erect, |
| | the whole tail drapes over your finger. |
| 2 | Limp tail and weakness of hind legs. |
| | When mouse is picked up by tail, legs are not spread apart, but |
| | held closer together. When the mouse is observed when walking, it |
| | has a clearly apparent wobbly walk. |
| 3 | Limp tail and complete paralysis of hind legs (most common). |
| | OR |
| | Limp tail with paralysis of one front and one hind leg. |
| | OR |
| | ALL of: |
| | Severe head tilting, |
| | Walking only along the edges of the cage, |
| | Pushing against the cage wall, |
| | Spinning when picked up by the tail. |
| 4 | Limp tail, complete hind leg and partial front leg paralysis. |
| | Mouse is minimally moving around the cage but appears alert and |
| | feeding. Usually, euthanasia is recommended after the mouse |
| | scores level 4 for 2 days. When the mouse is euthanized because |
| | of severe paralysis, score of 5 is entered for that mouse for |
| | the rest of the experiment. |
| 5 | Complete hind and complete front leg paralysis, no movement |
| | around the cage. |
| | OR |
| | Mouse is spontaneously rolling in the cage. |
| | OR |
| | Mouse is found dead due to paralysis. |

* In-between scores were assigned when the clinical signs fell between two above definedscores.

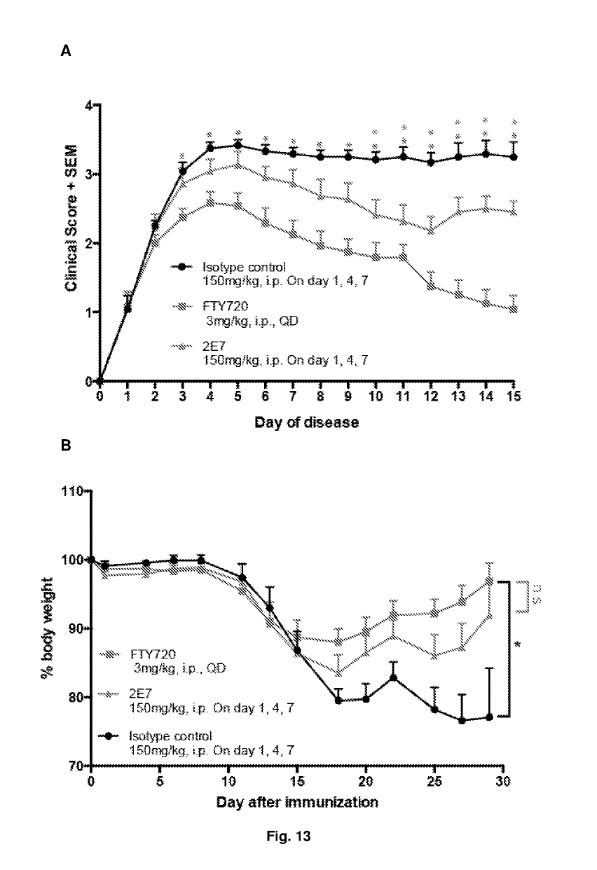

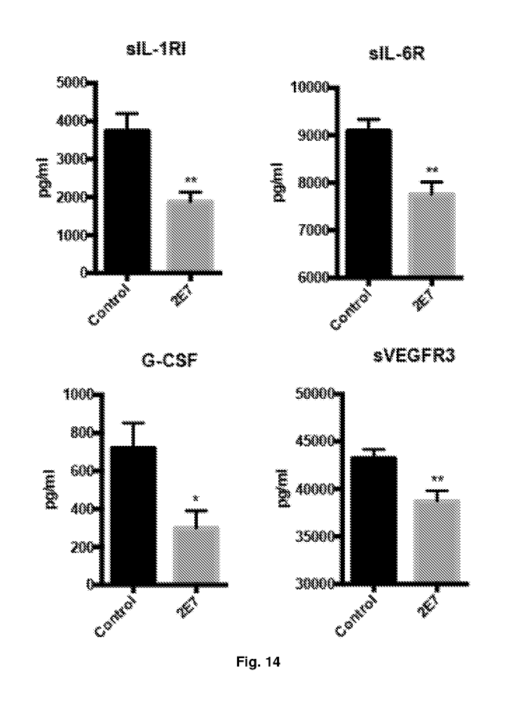

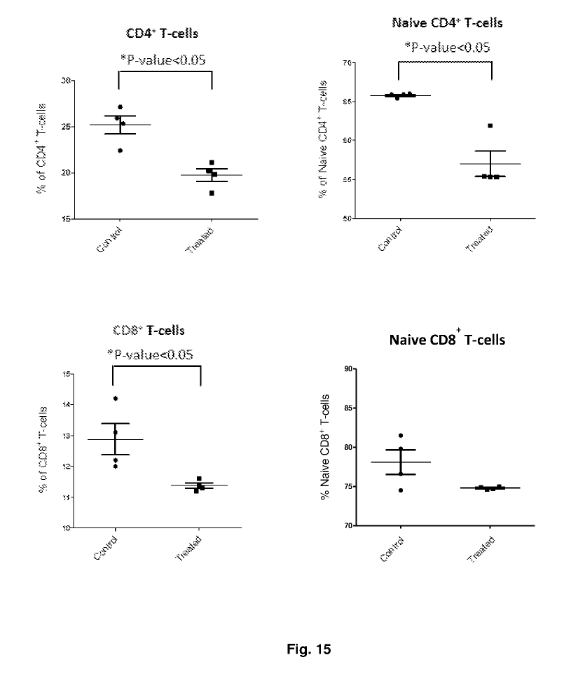

MS is the prototypical inflammatory demyelinating disease of the central nervous system (CNS) caused by autoimmunity. It is estimated to affect up to two million people worldwide. As 2E7 has shown efficacy in mouse model for rheumatoid arthritis, an autoimmune disease in joints, 2E7 was also tested in mouse EAE model, the most commonly used mouse model for human MS. As shown in FIG. 13, the clinical symptoms, paralysis of limbs and tail, and the loss of body weight of the mice were significantly improved after receiving three doses of 2E7 compared to mice receiving isotype control antibody. FTY720, a widely used small molecule drug, was used as a positive control in this study.

Monoclonal antibodies raised against N-acetylmuramyl-L-alanyl-D-isoglutamine are known in the art. One such antibody mAb2-4, isotype IgG2a, has been characterized in detail are known in the art. So far, in literature mAb2-4 has been used only in immunostaining of tissues to detect the presence of peptidoglycan mainly in inflammatory tissues and macrophages. However, there has been no report of applying this antibody to detect peptidoglycan or MPs in solution by ELISA. mAb2-4 were found to have very low affinity to MPs. Inhibition assays using mAb2-4 showed that 50% inhibition of mAb2-4 binding to peptidoglycan by N-acetylmuramyl-L-alanyl-D-isoglutamine occurred only at concentrations higher than 1 mg/ml, which is significantly lower than the picomolar affinity of the antibody of the present disclosure, 2E7 mAb.

Additionally, structural analysis of antigenic determinant showed that the mAb2-4 antibody recognizes the N-acetylmuramic acid linked to the dipeptide but not N-acetylmuramic acid or the dipeptide alone and that the N-acetyl group on muramic acid is an important antigenic determinant. Thus, the antigenic determinant on N-acetylmuramyl-L-alanyl-D-isoglutamine for mAb2-4 is different from that for the antibody of the present disclosure, (e.g. 2E7) which recognizes MDPs with or without the N-acetyl group. As many bacterial species do not have the N-acetyl group in the muramic acid residue, it follows that mAb2-4 has narrower specificity than 2E7 in recognizing bacterial species.

Another rather commonly used mouse monoclonal antibody for peptidoglycan is mAb2E9. This antibody was developed by immunizing mice with partially purified peptidoglycan-polysaccharide complexes isolated from feces of a healthy human. The affinity of this antibody to N-acetylmuramyl-L-alanyl-D-isoglutamine was found to be even lower than mAb2-4, and the antigenic determinant has not been defined. mAb2E9 has been used in immunostaining of tissues but never in ELISA.

Other monoclonal antibodies developed by immunizing mice with peptidoglycan isolated from Streptococcus mutans have also been described. Although these antibodies could recognize peptidoglycan prepared from multiple bacterial species including both Gram-positive and gram-negative ones, the binding could not be inhibited by N-acetylmuramyl-L-alanyl-D-isoglutamine and the antigenic determinant is not known.

In summary, currently available mouse monoclonal antibodies developed against either N-acetylmuramyl-L-alanyl-D-isoglutamine or peptidoglycan have limited use because of low affinity, poorly defined antigenic determinant and narrow specificity. None of these antibodies can be used to detect peptidoglycan or MPs in solution at sensitivity levels required by most research and clinical needs.

As shown in the present disclosure, 2E7 mAb can detect MPs with picomolar affinity. Furthermore, 2E7 recognizes an epitope universally present in all bacterial species. The antigenic determinant is formed with structural contributions from multiple molecular moieties and structural features only found in bacteria, ensuring the high specificity of 2E7 for bacterial peptidoglycan.

As shown in the present disclosure, 2E7 mAb neutralizes MDP containing molecules in circulation, thereby reduces the activation of intracellular pattern recognition receptor NOD2 and blocks the NOD2 signaling pathway. 2E7 mAb suppresses the development and progression of immune-mediated diseases such as rheumatoid arthritis by exerting its effect through a different pathway as compared to most conventional DMARDs. 2E7 mAb therefore provides a promising alternative therapeutic biologic for the treatment of immune-mediated diseases, especially for those patients that respond poorly to the therapeutic biologics currently available on the market.

In addition, the present disclosure shows that combination therapies comprising 2E7 mAb and one or more other therapeutic agents have a synergistic effect for the treatment of immune-mediated diseases compared to the monotherapies. Thus 2E7 mAb can be used in the development of combination therapies for the treatment of immune-mediated diseases such as rheumatoid arthritis to achieve more efficacious treatment results and/or to reduce the side effects of the currently available therapies.

1. A method of prophylactically or therapeutically treating an autoimmune or inflammatory disease comprising administering an isolated antibody or an antigen-binding fragment thereof, wherein said isolated antibody or antigen-binding fragment thereof is capable of binding to a muramyl peptide, or a derivative or an analog or a salt thereof, wherein said muramyl peptide comprises muramic acid and an amino acid selected from the group consisting of alanine, isoglutamine, glutamic acid, and a salt thereof.

2. The method according to claim 1, wherein said muramic acid has one or more of the following properties: comprises an N-acetyl group, and does not comprise an N-acetyl group.

3. (canceled)

4. The method according to claim 1, wherein said alanine is L-alanine, or a salt thereof.

5. The method according to claim 1, wherein said isoglutamine is D-isoglutamine, or a salt thereof.

6. The method according to claim 1, wherein said glutamic acid is D-glutamic acid, or a salt thereof.

7. The method according to claim 1, wherein said amino acid comprises one or more of the following: (a) L-alanine or a salt thereof, and D-isoglutamine or a salt thereof, and (b) L-alanine or a salt thereof, and D-glutamic acid or a salt thereof.

8. (canceled)

9. The method according to claim 1, wherein said muramyl peptide, or derivative or analog or salt thereof is one or more of the following: (a) selected from the group consisting of N-acetylmuramyl-L-alanyl-D-isoglutamine, muramyl-L-alanyl-D-isoglutamine, N-acetylmuramyl-L-alanyl-D-glutamate, and muramyl-L-alanyl-D-glutamate; and (b) part of a peptidoglycan or fragment thereof.

10. (canceled)

11. The method according to claim 1, wherein said isolated antibody or antigen-binding fragment thereof comprises one or more of the following: a heavy chain encoded by the nucleotide sequence of SEQ ID NO: 1, a light chain encoded by the nucleotide sequence of SEQ ID NO: 2, and a heavy chain encoded by the nucleotide sequence of SEQ ID NO: 1 and a light chain encoded by the nucleotide sequence of SEQ ID NO: 2.

12. (canceled)

13. (canceled)

14. The method according to claim 1, wherein said isolated antibody or antigen-binding fragment thereof comprises one or more of the following: a heavy chain variable domain comprising the amino acid sequence as set forth in SEQ ID NO: 3, or a variant thereof; an amino acid sequence having at least 70%, at least 75%, at least 80%, at least 85%, at least 90%, at least 95%, at least 96%, at least 97%, at least 98%, or at least 99% identity with the amino acid sequence as set forth in SEQ ID NO: 3; a light chain variable domain comprising the amino acid sequence as set forth in SEQ ID NO: 4, or a variant thereof; an amino acid sequence having at least 70%, at least 75%, at least 80%, at least 85%, at least 90%, at least 95%, at least 96%, at least 97%, at least 98%, or at least 99% identity with the amino acid sequence as set forth in SEQ ID NO: 4; and a heavy chain variable domain comprising the amino acid sequence as set forth in SEQ ID NO: 3, or a variant thereof, and a light chain variable domain comprising the amino acid sequence as set forth in SEQ ID NO: 4, or a variant thereof.

15.-18. (canceled)

19. The method according to claim 1, wherein said isolated antibody or antigen binding fragment thereof is monoclonal.

20. The method according to claim 19, wherein said monoclonal antibody is of the subtype IgG1.

21. The method according to claim 19, wherein said monoclonal antibody is selected from the group consisting of a humanized antibody and a chimeric antibody.

22. (canceled)

23. The method according to claim 1, wherein said isolated antibody or antigen-binding fragment binds to said muramyl peptide, or a derivative or an analog or a salt thereof, with a Kd value selected from the group consisting of less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, and less than about 10 pM.

24. (canceled)

25. (canceled)

26. The method according to claim 1, wherein the autoimmune or inflammatory disease is selected from the group consisting of sepsis, septic shock, Crohn's disease, rheumatoid arthritis, asthma, allergy, atopic disorders, multiple sclerosis, pertussis, gonorrhea, inflammatory bowel disease, and antibiotic-associated disorder.

27. (canceled)

28. (canceled)

29. A composition comprising an isolated antibody or an antigen-binding fragment thereof as defined in claim 1, one or more therapeutic agents, and optionally a pharmaceutically acceptable carrier.

30. The composition according to claim 29, wherein said one or more therapeutic agents are selected from the group consisting of nonsteroidal anti-inflammatory drugs (NSAIDs), non-biologic and biologic disease-modifying anti-rheumatic drugs (DMARDs), immunosuppressants, and corticosteroids.

31. The composition according to claim 30, wherein said DMARD is selected from the group consisting of methotrexate, hydroxychloroquine, sulfasalazine, leflunomide, Tumor Necrosis Factor (TNF) inhibitors, T-cell costimulatory blocking agents, B cell depleting agents, Interleukin-6 (IL-6) inhibitors and Interleukin-1 (IL-1) receptor antagonists.

32. The composition according to claim 31, wherein said TNF inhibitor is selected from the group consisting of etanercept, adalimumab, infliximab, certolizumab pegol and golimumab.

33. A method of prophylactically or therapeutically treating an autoimmune or inflammatory disease comprising administering a composition of claim 29.

34. (canceled)

35. (canceled)

36. The method according to claim 33, wherein the autoimmune or inflammatory disease is selected from the group consisting of sepsis, septic shock, Crohn's disease, rheumatoid arthritis, asthma, allergy, atopic disorders, multiple sclerosis, pertussis, gonorrhea, inflammatory bowel disease, and antibiotic-associated disorder.

37. (canceled)

38. (canceled)
